(19) United States
(12) Patent Application Publication
(10) Pub. No.: US 2025/0065136 A1
(43) Pub. Date: Feb. 27, 2025

(54) Device and Method for Current Steering in an Implantable Device by an External Generator

(71) Applicant: Exact Neuro LLC, Napa, CA (US)

(72) Inventor: Arkady Glukhovsky, Santa Clarita, CA (US)

(21) Appl. No.: 18/726,399

(22) PCT Filed: Jan. 2, 2023
(86) PCT No.: PCT/US23/60006

§ 371 Date: Jul. 2, 2024

Related U.S. Application Data
(60) Provisional application No. 63/295,907, filed on Jan. 2, 2022.

(51) Int. Cl. A61N 1/378; A61N 1/05; A61N 1/372

(52) U.S. Cl. CPC A61N 1/3787; A61N 1/0504; A61N 1/37247

(57) ABSTRACT

An external generator applies a burst of transcutaneous current including an alternating current component and an offset component to the skin of a patient. An implantable device including a first channel and a second channel is implanted beneath the skin. A portion of the burst of transcutaneous current passes through the one or more implantable device implanted beneath the skin of the subject. The first channel includes a rectifier allowing positive current to pass through the first channel. The second channel includes a rectifier allowing negative current to pass through the second channel. The burst of transcutaneous current delivers a first electrical charge to tissue at an output electrode of the first channel based on the offset component. The burst of transcutaneous current delivers a second electrical charge different than the first electrical charge to tissue at an output electrode of the second channel based on the offset component.

### CROSS REFERENCE TO RELATED APPLICATIONS

This patent application claims the benefit of and priority to U.S. Provisional Patent Application Ser. No. 63/295,907, entitled DEVICE AND METHOD FOR ELECTRICAL STIMULATION, filed Jan. 2, 2022, the contents of which are hereby incorporated by reference in their entirety. This application is related to co-pending International Patent Application Serial No.__/______, entitled DEVICE AND METHOD FOR DELIVERY OF TRANSCUTANOUS CURRENT, filed contemporaneously herewith on Jan. 2, 2023, and co-pending International patent application Serial No.__/______, entitled DEVICE AND METHOD FOR EVALUATING COUPLING WITH AN IMPLANTABLE DEVICE, filed contemporaneously herewith on Jan. 2, 2023, the contents of which are hereby incorporated by reference in their entirety.

### BACKGROUND

### Technical Field

The present disclosure relates generally to medical devices and treatments, and more particularly, to a partially implantable system for delivery of transcutaneous current.

### Introduction

Implantable devices can be implanted into tissue of a subject, such as a human patient, to apply electrical current to nerves or tissue (e.g., to stimulate nerves or tissue) and to record or sense neural signals emanating from the central and peripheral nervous systems. Implantable devices can be used for a variety of clinical indications, including delivering electrical stimulation and/or sensing to treat pain, incontinence, motor response, headache and migraine, and other indications.

Some systems may use an external device, such as an external generator, to apply transcutaneous to the skin of a subject and use an implantable device beneath the skin of the subject to provide a path for the applied transcutaneous current. An implantable device may include multiple electrodes for delivery of current. It may be desirable to control the current delivered to each of the multiple electrodes. It may also be desired to minimize the size of the implantable device, so it may not be desirable to include components and/or utilize power for routing current from inside the implantable device.

### SUMMARY

The following presents a simplified summary of one or more aspects in order to provide a basic understanding of such aspects. This summary is not an extensive overview of all contemplated aspects, and is intended to neither identify key or critical elements of all aspects nor delineate the scope of any or all aspects. Its sole purpose is to present some concepts of one or more aspects in a simplified form as a prelude to the more detailed description that is presented later.

In an aspect of the disclosure, a method of delivering transcutaneous current to tissue of a subject through a first channel and a second channel of one or more implantable device implanted beneath the skin of the subject is provided. The method may be performed by an external generator. The method may include applying a burst of transcutaneous current to the skin of the subject, the burst of transcutaneous current comprising an alternating current component and an offset component, wherein a portion of the burst of transcutaneous current passes through the one or more implantable device implanted beneath the skin of the subject, the first channel comprises a rectifier allowing positive current to pass through the first channel, the second channel comprises a rectifier allowing negative current to pass through the second channel, the burst of transcutaneous current delivers a first electrical charge to tissue at an output electrode of the first channel based on the offset component, and the burst of transcutaneous current delivers a second electrical charge different than the first electrical charge to tissue at an output electrode of the second channel based on the offset component.

In some aspects, the first electrical charge activates the tissue at the output electrode of the first channel and the second electrical charge does not activate the tissue at the output electrode of the second channel.

In some aspects, the first electrical charge activates the tissue at the output electrode of the first channel, the second electrical charge activates the tissue at the output electrode of the second channel, and wherein the activation level of the tissue at the output electrode of the first channel is different than the activation level of the tissue at the output electrode of the second channel.

In some aspects, the offset component is applied for the entire duration of the alternating current component.

In some aspects, the offset component is applied for a subset of the duration of the alternating current component.

In some aspects, the offset component is applied before the alternating current component.

In some aspects, the offset component is applied after the alternating current component.

In some aspects, one implantable device comprises the first stimulation channel and the second stimulation channel.

In some aspects, a first implantable device comprises the first channel and a second implantable device comprises the second channel.

In some aspects, the method includes receiving a first input corresponding to a first current level for the first channel; and receiving a second input corresponding to a second current level for the second channel, wherein a characteristic of the offset component and a characteristic of the alternating current component are based on the first input and the second input.

In some aspects, the characteristic of the alternating current component is an amplitude of the alternating current component and the characteristic of the offset component is an amplitude, a shape, or a duration of the offset component.

In some aspects, the method includes applying a second burst of transcutaneous current to the skin of the subject, the second burst of transcutaneous current comprising a second alternating current component and a second offset component, wherein the second burst of transcutaneous current delivers a third electrical charge to the tissue at the output electrode of the first channel based on the second offset component, the second burst of transcutaneous current delivers a fourth electrical charge to the tissue at the output electrode of the second channel based on the second offset component, the first electrical charge is greater than the second electrical charge, and the fourth electrical charge is greater than the third electrical charge.

In some aspects, the offset component and the second offset component are charge balanced.

In some aspects, the method includes applying a balancing current to the skin of the subject, wherein the balancing current is charge balanced with the offset component.

In some aspects, the balancing current is not applied during the burst of transcutaneous current.

In some aspects, the balancing current has a longer duration than the offset component.

In some aspects, the method includes receiving an input from a user interface or a sensor, wherein a characteristic of the offset component is based on the input.

In some aspects, the characteristic is an amplitude, a shape, a timing, or a duration of the offset component.

In another aspect of the disclosure, an apparatus is provided. The apparatus may be an external generator. The external generator may include a current generator, a first skin electrode coupled to the current generator, and a second skin electrode coupled to the current generator. The current generator may be configured to apply a burst of transcutaneous current between the first skin electrode and the second skin electrode to a skin of a subject, the burst of transcutaneous current comprising an alternating current component and an offset component, a portion of the burst of transcutaneous current passes through one or more implantable device implanted beneath the skin of the subject, the one or more implantable device including a first channel and a second channel, the first channel of the implantable device comprises a rectifier allowing positive current to pass through the first channel, the second channel comprises a rectifier allowing negative current to pass through the second channel, the burst of transcutaneous current delivers a first electrical charge to tissue at an output electrode of the first channel based on the offset component, and the burst of transcutaneous current delivers a second electrical charge different than the first electrical charge to tissue at an output electrode of the second channel based on the offset component.

In some aspects, the first electrical charge activates the tissue at the output electrode of the first channel and the second electrical charge does not activate the tissue at the output electrode of the second channel.

In some aspects, the first electrical charge activates the tissue at the output electrode of the first channel, the second electrical charge activates the tissue at the output electrode of the second channel, and wherein the activation level of the tissue at the output electrode of the first channel is different than the activation level of the tissue at the output electrode of the second channel.

In some aspects, the current generator is configured to apply the offset component for the entire duration of the alternating current component.

In some aspects, the current generator is configured to apply the offset component for a subset of the duration of the alternating current component.

In some aspects, the current generator is configured to apply the offset component before the alternating current component.

In some aspects, the current generator is configured to apply the offset component after the alternating current component.

In some aspects, one implantable device comprises the first stimulation channel and the second stimulation channel.

In some aspects, a first implantable device comprises the first channel and a second implantable device comprises the second channel.

In some aspects, the external generator includes a controller configured to receive a first input corresponding to a first current level for the first channel; and receive a second input corresponding to a second current level for the second channel, wherein a characteristic of the offset component applied by the current generator and a characteristic of the alternating current component applied by the current generator based on the first input and the second input.

In some aspects, the characteristic of the alternating current component is an amplitude of the alternating current component and the characteristic of the offset component is an amplitude, a shape, or a duration of the offset component.

In some aspects, the current generator is further configured to apply a second burst of transcutaneous current to the skin of the subject, the second burst of transcutaneous current comprising a second alternating current component and a second offset component, wherein the second burst of transcutaneous current delivers a third electrical charge to the tissue at the output electrode of the first channel based on the second offset component, the second burst of transcutaneous current delivers a fourth electrical charge to the tissue at the output electrode of the second channel based on the second offset component, the first electrical charge is greater than the second electrical charge, and the fourth electrical charge is greater than the third electrical charge.

In some aspects, the offset component and the second offset component are charge balanced.

In some aspects, the current generator is further configured to apply a balancing current to the skin of the subject, wherein the balancing current is charge balanced with the offset component.

In some aspects, the current generator does not apply the balancing current during the burst of transcutaneous current.

In some aspects, the balancing current has a longer duration than the offset component.

In some aspects, the external generator includes a controller configured to receive an input from a user interface or a sensor, wherein a characteristic of the offset component is based on the input.

In some aspects, the characteristic is an amplitude, a shape, a timing, or a duration of the offset component.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

FIG. 1 is a diagram illustrating a system for applying transcutaneous current.

FIG. 2 is a circuit diagram of an implantable device.

FIG. 3 is a diagram illustrating a transcutaneous current applied by an external generator, current at an output electrode of a first channel, and current at an output electrode of a second channel.

FIG. 4 is a diagram illustrating transcutaneous current with alternating current components and offset components.

FIG. 5 is a diagram illustrating the current at the output electrodes of an implantable device when the transcutaneous current of FIG. 4 is applied to the skin of a subject.

FIG. 6 is a diagram illustrating a burst of transcutaneous current including an offset component with a net positive sum of charge that begins before the first time t1 and ends at the first time t1.

FIG. 7 is a diagram illustrating a burst of transcutaneous current including an offset component with a net positive sum of charge that begins before the first time t1 and ends after the first time t1 but before the second time t2.

FIG. 8 is a diagram illustrating a burst of transcutaneous current including an offset component with a net positive sum of charge that begins after the first time t1 but before the second time t2 and ends after the second time t2.

FIG. 9 is a diagram illustrating a burst of transcutaneous current including an offset component with a net positive sum of charge that begins before the first time t1 and ends after the first time t2.

FIG. 10 is a diagram illustrating a burst of transcutaneous current including an offset component with a net positive charge that begins after the first time t1 and ends before the first time t2.

FIG. 11 is a diagram illustrating a burst of transcutaneous current including an offset component with a net positive charge.

FIG. 12 is a diagram illustrating bursts of transcutaneous current including charge balanced offset components.

FIG. 13 is a diagram illustrating transcutaneous current with a balancing current.

FIG. 14 is a block diagram illustrating an external generator.

FIG. 15 is a flowchart illustrating a method of delivering transcutaneous current to tissue of a subject through a first channel and a second channel of one or more implantable device.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components may be shown in block diagram form in order to avoid obscuring such concepts.

Several aspects of medical devices will now be presented with reference to various apparatus and methods.

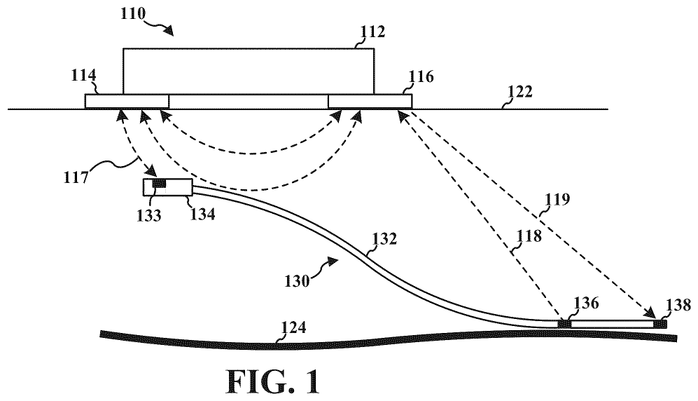

FIG. 1 is a diagram illustrating a system for applying transcutaneous current. The system includes an external generator 110 and an implantable device 130.

The implantable device 130 includes a receiving electrode 133, a first output electrode 136, a second output electrode 138, an electronics housing 134, and a lead 132. The implantable device 130 may be implanted beneath the skin 122 of the subject. The first output electrode 136 and the second output electrode 138 may be implanted at locations targeted for delivery of current. For example, in some aspects, the first output electrode 136 may be implanted in proximity to a first location on a nerve 124, and the second output electrode 138 may be implanted in proximity to a second location on the nerve 124. In some aspects, the first output electrode 136 may be implanted in proximity to a first nerve and the second output electrode 138 may be implanted in proximity to a second nerve. In some aspects, the first output electrode 136 and the second output electrode 138 may be stimulating electrodes. They may be implanted at locations targeted for delivery of stimulation by the system for applying transcutaneous current. The current passing through the first output electrode 136 and the second output electrode 138 may stimulate the tissue surrounding the electrodes (e.g., may activate a nerve at the implantation site of the electrode).

The external generator 110 includes a device housing 112, a source electrode 114, and a return electrode 116. The source electrode 114 and the return electrode 116 may be electrodes configured to make electrical contact with the skin 122 of a subject. The source electrode 114 and the return electrode 116 may include hydrogel, wetted cloth, metal, conductive polymer, or other materials. The source electrode 114 and the return electrode 116 may be attached directly to the device housing 112, or one or both may be attached to the device housing 112 by a lead or cable.

The source electrode 114 and the return electrode 116 may be similar or identical electrodes attached to opposite ends of the current generating circuit: the names “source” and “return” are used herein for clarity to differentiate where the electrode is placed with respect to the implantable device, and to clarify the meaning of “positive” and “negative” current in a system for applying transcutaneous current. The source electrode 114 may be the electrode placed above the implantation site of a receiving electrode of the implantable device, and the return electrode 116 may be the electrode placed at a different location (e.g., above or near the implantation site of the output electrodes of the implantable device). Positive current, as used herein, refers to current passing from the source electrode (e.g., the electrode proximate the receiving electrode) to the return electrode (e.g., the electrode placed at a different location), and negative current, as used herein, refers to current passing from the return electrode to the source electrode. In some aspects, the electrodes of the external generator 110 may be interchangeable and whichever electrode is placed above the receiving electrode of the implantable device may be referred to as the source electrode as used herein.

The implantable device 130 includes two channels. A channel may be an electrical path for current to pass through the implantable device 130. The implantable device 130 includes a first channel between the receiving electrode 133 and the first output electrode 136. The implantable device 130 includes a second channel between the receiving electrode 133 and the second output electrode 138. In some aspects, each channel may be coupled to a single output electrode, as shown in FIG. 1. In some aspects, a channel can be coupled to multiple output electrodes (e.g., a first channel may be coupled to a first output electrode and a second output electrode and a second channel may be coupled to a third output electrode and a fourth output electrode.

The source electrode 114 may be placed in contact with the skin 122 of the subject near or directly over the implantation site of the receiving electrode 133. The return electrode 116 may be placed in contact with the skin 122 of the subject spaced apart from the source electrode 114. For example, the return electrode 115 may be placed near or directly over the implantation site of the first output electrode 136 and/or the second output electrode 138.

The external generator 110 may apply transcutaneous alternating current to the skin 122 of the subject, which may flow between the source electrode 114 and the return electrode 116 through the skin 122 and subcutaneous tissue of the subject. In some aspects, the external generator 110 may apply bursts of transcutaneous alternating current to the skin 122 of the subject. Current flowing from the source electrode 114 to the return electrode 116 may be referred to herein as positive current, and current flowing from the return electrode 116 to the source electrode 114 may be referred to herein as negative current. The channels of the implantable device 130 may provide a path for transcutaneous current to pass through the implantable device 130. A portion of the transcutaneous current applied by the external generator 110 may pass through the channels of the implantable device 130. The lower the impedance of the channel relative to the impedance of the surrounding tissue, the larger the portion of the transcutaneous current flowing via the channel of the implantable device 130. The portion of the transcutaneous current passing through a channel may deliver electrical charge to the tissue surrounding the output electrode of that channel. The electrical charge may stimulate the tissue.

The channels of the implantable device 130 may rectify transcutaneous current passing through the channels. The implantable device 130 may include one channel which allows positive current to pass through the implantable device 130 (e.g., configured for anodic stimulation) and another channel which allows negative current to pass through the implantable device 130 (e.g., configured for cathodic stimulation). For example, as shown in FIG. 1, the first channel between the receiving electrode 133 and the first output electrode 136 may rectify transcutaneous current to allow current to pass from the first output electrode 136 to the return electrode 116 but to inhibit or block current passing from the return electrode 116 to the first output electrode 136 (e.g, the first channel may be configured for anodic stimulation). The second channel between the receiving electrode 133 and the second output electrode 138 may rectify transcutaneous current to allow current to pass from the return electrode 116 to the second output electrode 138 but to inhibit or block current passing from the second output electrode 138 to the return electrode 116 (e.g., the second channel may be configured for cathodic stimulation).

In some aspects, the external generator 110 may apply bursts of transcutaneous current including alternating current components and offset components to implement current steering between the first output electrode 136 and the second output electrode 138. The external generator 110 may apply a burst of transcutaneous current with an offset component with a net positive charge to deliver more current through the first channel to the first output electrode 136 and less current through the second channel to the second output electrode 138. The external generator 110 may apply a burst of transcutaneous current with an offset component with a net negative charge to delivery more current through the second channel to the second output electrode 138 and less current through the first channel to the first output electrode 136. In some aspects, the external generator 110 may include a user interface, and may receive inputs from the user interface (or may receive user input from a remote device) and may base the amplitude, shape, timing, or other characteristics of an offset component based on the inputs. In some aspects, the external generator 110 may include one or more sensor which provides the external generator 110 with sensor input, or may receive sensor input from a remote device, and may base the amplitude, shape, timing, or other characteristics of an offset component based on the sensor input.

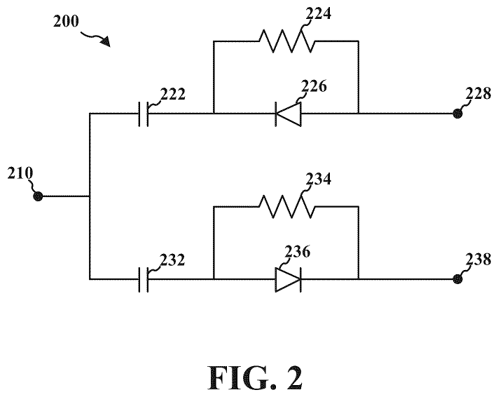

FIG. 2 is a circuit diagram 200 of an implantable device. The implantable device includes a receiving electrode 210, a first output electrode 228, and a second output electrode 238. The implantable device includes a first channel between the receiving electrode 210 and the first output electrode 228 and includes a second channel between the receiving electrode 210 and the second output electrode 238. The first channel includes a first capacitor 222, a first diode 226, and a first resistor 224 in parallel with the first diode 224. The first diode 226 is oriented to rectify current in the first channel, allowing negative current (e.g., current flowing from the first output electrode 228 to the receiving electrode 210) to pass through the first channel (e.g., the first channel is configured for cathodic stimulation). The second channel includes a second capacitor 232, a second diode 236, and a second resistor 234 in parallel with the second diode 236. The second diode 236 is oriented to rectify current in the second channel, allowing positive current (e.g., current flowing from the receiving electrode 210 to the second output electrode 238) to pass through the second channel (e.g., the second channel is configured for anodic stimulation).

When an external generator applies transcutaneous current to the tissue surrounding the implantable device, a portion of the applied current may pass through the implantable device.

In some aspects, an implantable device may include additional electrical components. For example, an implantable device may include an electrostatic discharge protection component in one or both channels. In some aspects, an implantable device may include fewer components than described with respect to FIG. 2. For example, an implantable device may not include the first resistor 224 and/or the second resistor 234. In some aspects, an implantable device may use another rectification element in place of the first diode 226 and/or may use another rectification element in place of the second diode 236. For example, an implantable device may utilize a transistor as a rectification element.

The implantable device 200 illustrated in FIG. 2 includes two channels. In some aspects, a system for applying transcutaneous stimulation according to the present disclosure may include multiple implants with different channels. For example, a system for applying transcutaneous stimulation may include one implantable device with a channel configured for anodic stimulation and another implantable device with a channel configured for cathodic stimulation. The two implantable devices may be implanted with their respective receiving electrodes in close proximity to one another. A circuit diagram illustrating the two implantable devices may resemble the circuit diagram illustrated in FIG. 2, but with each channel coupled to a separate receiving electrode. However, as the two receiving electrodes may be in close proximity to one another, the overall system would function similarly to the description provided above for the implantable device 200 of FIG. 2.

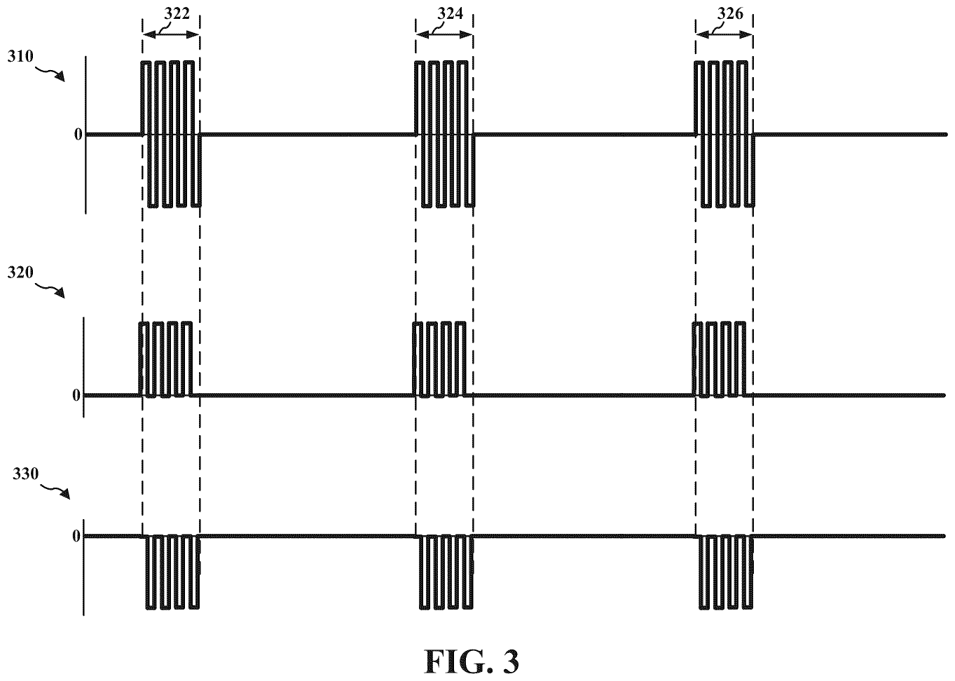

FIG. 3 is a diagram illustrating a transcutaneous current applied by an external generator 310, current at an output electrode of a first channel 320, and current at an output electrode of a second channel 330. An external generator may apply transcutaneous current 310 to the skin of a subject. For example, the external generator may be the external generator 110 of FIG. 1. One or more implantable device may be implanted beneath the skin of a subject. A portion of the transcutaneous current 310 applied by the external generator may pass through the one or more implantable device. The portion of the transcutaneous current 310 passing through the one or more implantable device may have the shape of the transcutaneous current 310 but a lower amplitude.

The transcutaneous current 310 includes a series of bursts of alternating transcutaneous current, including a first burst of alternating transcutaneous current 322, a second burst of alternating transcutaneous current 324, and a third burst of alternating transcutaneous current 326. Each burst of alternating transcutaneous current includes high frequency current pulses of alternating polarity (e.g., voltage or current which alternates between positive and negative at a high frequency). The positive and negative current pulses may have the same amplitude. The bursts of transcutaneous stimulation may be symmetrical with respect to the amplitude of the burst.

The one or more implantable device beneath the skin of the subject may include a first channel and a second channel. In some aspects, a single implantable device may include both the first channel and the second channel. For example, the one or more implantable device may be the implantable device 130 of FIG. 1. In some aspects, a first implantable device may include the first channel; and, a second implantable device may include the second channel.

The first channel may inhibit current passing through the first channel in one direction, and the second channel may inhibit current passing through the second channel in another direction. For example, the first channel may allow positive current to pass through the channel but may prevent or inhibit negative current from passing through the first channel, and the second channel may allow negative current to pass through the channel but may prevent or inhibit positive current from passing through the second channel. The first channel and the second channel may include rectification components, for example, as described above with respect to the first diode 226 and the second diode 236 of FIG. 2.

The first channel current 320 is the current at the output electrode of the first channel when the transcutaneous current 310 is applied by the external generator. The second channel current 330 is the current at the output electrode of the second channel when the transcutaneous current 310 is applied by the external generator. The first channel current 320 includes the positive portion of the current that passes through the implantable device and the second channel current 330 includes the negative portion of the current that passes through the implantable device.

The first channel and the second channel may have the same or similar impedance in opposite directions. As the transcutaneous current 310 applied by the external generator consists of pulses of alternating polarity current, the first channel current 320 and the second channel current 330 may deliver the same or similar levels of opposite polarity charge to the tissue at the output electrode of the first channel and the tissue at the output electrode of the second channel. In some aspects, it may be desirable for a system for applying transcutaneous current to deliver disparate levels of charge to the tissue at the output electrodes of the first channel and the second channel.

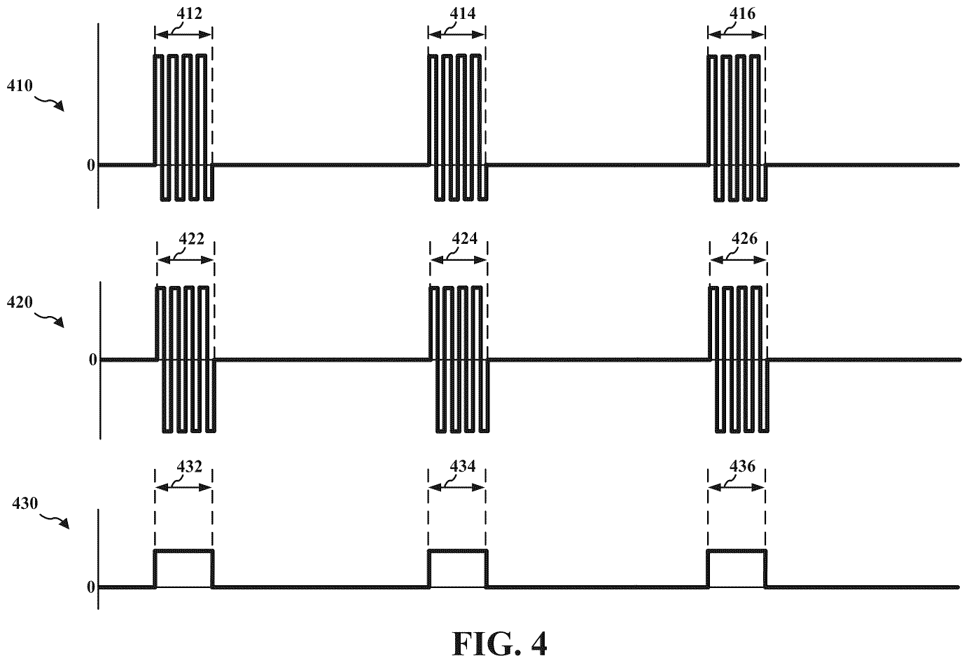

FIG. 4 is a diagram illustrating transcutaneous current with alternating current components and offset components. An external generator, such as the external generator 110 of FIG. 1, may apply transcutaneous current 410 to the skin of a subject. The transcutaneous current 410 includes a set of bursts of transcutaneous current, including a first burst of transcutaneous current 412, a second burst of transcutaneous current 414, and a third burst of transcutaneous current 416.

The transcutaneous current 410 may be represented as the sum of a set of alternating current components 420 and a set of offset components 430. The first burst of transcutaneous current 412 may be represented as the sum of a first alternating current component 422 and a first offset component 432. The second burst of transcutaneous current 414 may be represented as the sum of a second alternating current component 424 and a second offset component 434. The third burst of transcutaneous current 416 may be represented as the sum of a third alternating current component 426 and a third offset component 436. In some aspects, the external generator may generate the alternating current components 420 and the offset components 430 separately and may add the two current waveforms together (e.g., using an adder circuit) to generate the transcutaneous current 410. In some aspects, the external generator may generate the transcutaneous current 410 directly.

Alternating current components may be pulses of current of the same amplitude alternating between positive and negative polarity. An alternating current component may have a frequency between tens of KHz to above one MHz. In some aspects, an alternating current component may include alternating rectangular pulses, as shown in FIG. 4. In some aspects, an alternating current component may have another shape, such as a sinusoidal wave, a sawtooth wave, a triangle wave, a trapezoidal wave, or an arbitrary waveform. Moreover, the first alternating current component 422, the second alternating current component 424, and the third alternating current component 426 may have different parameters such as different frequency, duration, and waveform. The time period between the first alternating current component 422 and the second alternating current component 424 and the time period between the second alternating current component 424 and the third alternating current component 426 may be different and/or may vary over the duration of the alternating current component. The first offset component 432, the second offset component 434, and the third offset component 436 may have different duration, amplitude, and shape, which may vary with over the duration of the offset component. The time period between the first offset component 432 and the second offset component 434 and the time period between the second offset component 434 and the third offset component 436 may be different and/or may vary.

Offset components may refer to waveforms with net positive or net negative sums of charge which, when combined with the alternating current components to form the bursts of transcutaneous current in the transcutaneous current 410, result in the corresponding burst having a net positive or net negative sum of charge. For example, the first offset component 432, the second offset component 434, and the third offset component 436 all have net positive sums of charge over their durations. In some aspects, an offset component may be a square pulse as illustrated in FIG. 4. In other aspects, an offset pulse may have other shapes that deliver a net positive or net negative sum of charge over the duration of the offset component.

The alternating current components 420 may be symmetrical with respect to their amplitudes, and the offset components 430 may be asymmetrical. The bursts of transcutaneous current may therefore be asymmetrical with respect to the amplitude of the burst based on the inclusion of the offset component.

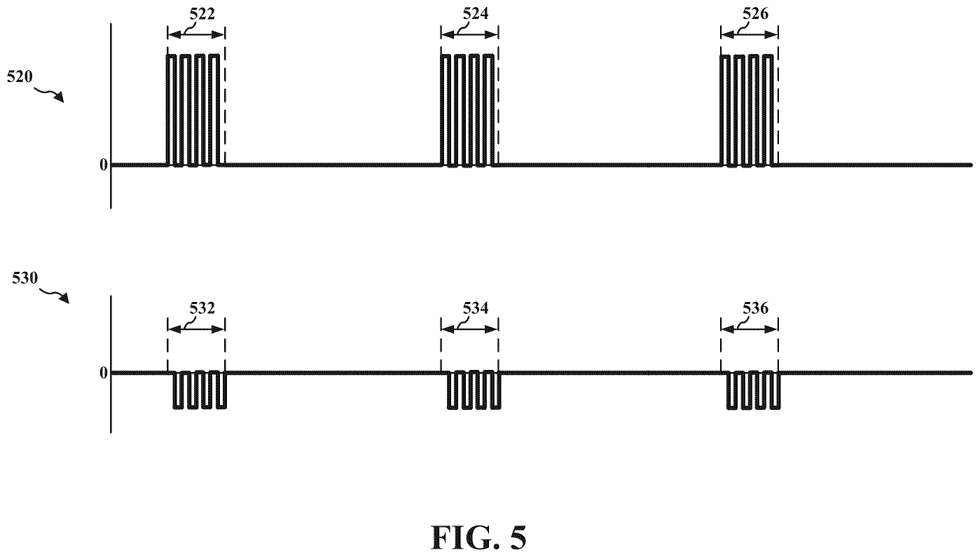

FIG. 5 is a diagram illustrating the current at the output electrodes of an implantable device when the transcutaneous current 410 of FIG. 4 is applied to the skin of a subject. The first channel of the implantable device may rectify the current to allow the positive portions of the current to pass through the first channel, resulting in first channel current 520 at the output electrode of the first channel, and the second channel of the implantable device may rectify the current to allow the negative portions of the current to pass through the second channel, resulting in second channel current 530 at the output electrode of the second channel.

The first channel current 520) includes a first burst of current 522 corresponding to the first burst of transcutaneous current 412, a second burst of current 524 corresponding to the second burst of transcutaneous current 414, and a third burst of current 526 corresponding to the third burst of transcutaneous current 416. The second channel current 530) includes a first burst of current 532 corresponding to the first burst of transcutaneous current 412, a second burst of current 534 corresponding to the second burst of transcutaneous current 414, and a third burst of current 536 corresponding to the third burst of transcutaneous current 416.

Electrical charge delivered to tissue may activate the tissue (e.g., the electrical charge may cause a difference between an intracellular and an extracellular electrical potential of the tissue, resulting in activation of the tissue). The first burst of transcutaneous current 412, the second burst of transcutaneous current 414, and the third burst of transcutaneous current 416 each include a net positive charge over their duration based on the net positive charge of the first offset component 432, the second offset component 434, and the third offset component 436. When the first channel and the second channel rectify the portion of the transcutaneous current 410 passing through the one or more implantable device, disparate levels of charge are delivered to the respective output electrodes of the channels. The bursts of the first channel current 520 include a series of pulses of positive current and the burst of the negative channel current 530 include a series of pulse of negative current, but the pulses in the first channel current 520 have a higher amplitude than the pulses in the second channel current 530). In some aspects, the electrical charge delivered to the tissue near the first output electrode of the first channel by the first channel current 520 may activate the tissue near the first output electrode, and the electrical charge delivered to the tissue near the second output electrode of the second channel by the second channel current 530 may not activate the tissue near the second output electrode. In some aspects, the electrical charge delivered to the tissue near the first output electrode of the first channel by the first channel current 520 may activate the tissue near the first output electrode, and the electrical charge delivered to the tissue near the second output electrode of the second channel by the second channel current 530 may activate the tissue near the second output electrode, but the activation of the tissue near the output electrode for the first channel may be greater or more intense than the activation of the tissue near the output electrode for the second channel.

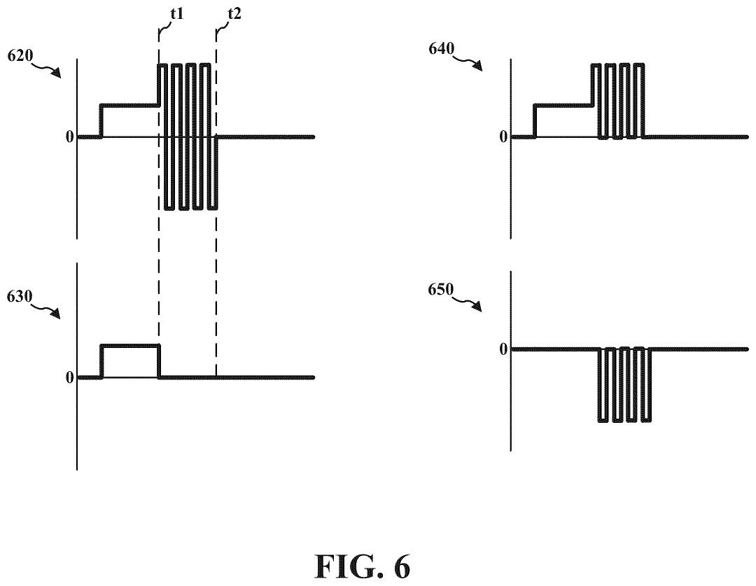

In some aspects, as illustrated in FIGS. 4 and 5, an offset component of a burst of transcutaneous current may begin and end at the same time as the corresponding alternating current component of the burst of transcutaneous current. In some aspects, the offset component may begin and end at different times than the alternating current component. An offset component may offset the charge delivered to respective output electrodes, but may not necessarily offset the alternating current component of the burst of transcutaneous current. FIGS. 6-10 illustrate bursts of transcutaneous current with an alternating current component beginning at a first time t1 and a second time t2, with an offset component beginning or ending at a different time, and their corresponding first channel currents and second channel currents.

In some aspects, an offset component of a burst of transcutaneous current may precede or follow the alternating current component. FIG. 6 is a diagram illustrating a burst of transcutaneous current 620 including an offset component 630 with a net positive sum of charge that begins before the first time t1 and ends at the first time t1. The corresponding first channel current 640 delivers a larger magnitude of charge than the corresponding second channel current 650 based on the offset component 630. In some aspects, the offset component may end at the same time that the alternating current component begins, as illustrated in FIG. 6. In some aspects, the offset component may end before the alternating current component begins. In some aspects, the offset component may begin at the same time that the alternating current component ends. In some aspects, the offset component may begin after the alternating current component ends.

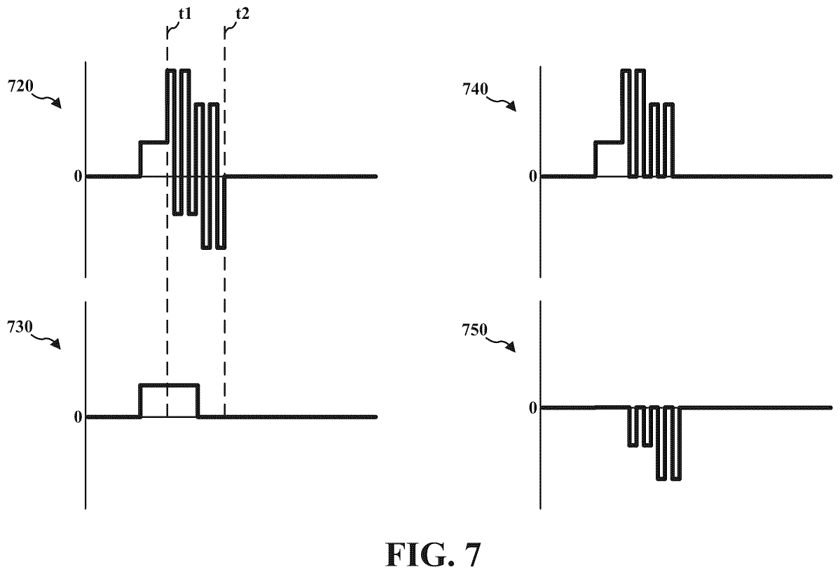

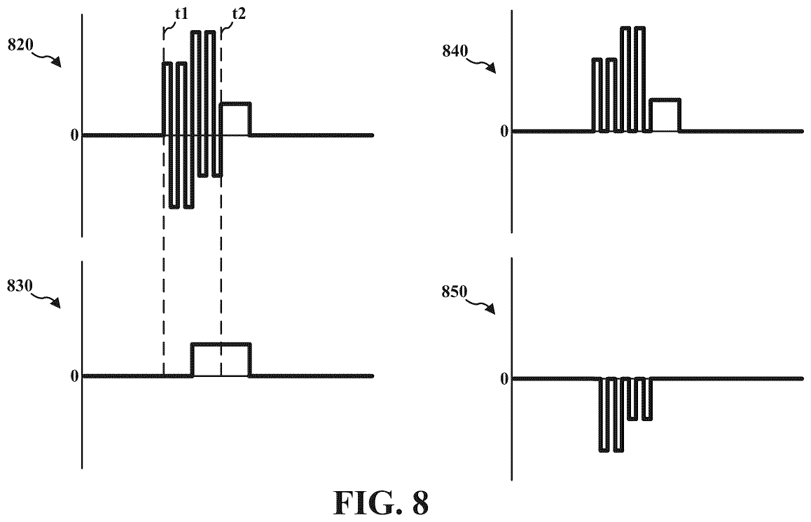

In some aspects, an offset component of a burst of transcutaneous current may partially overlap the alternating current component. FIG. 7 is a diagram illustrating a burst of transcutaneous current 720 including an offset component 730 with a net positive sum of charge that begins before the first time t1 and ends after the first time t1 but before the second time t2. The corresponding first channel current 740 delivers a larger magnitude of charge than the corresponding second channel current 750 based on the offset component 730. FIG. 8 is a diagram illustrating a burst of transcutaneous current 820 including an offset component 830 with a net positive sum of charge that begins after the first time t1 but before the second time t2 and ends after the second time t2. The corresponding first channel current 840 delivers a larger magnitude of charge than the corresponding second channel current 850 based on the offset component 830.

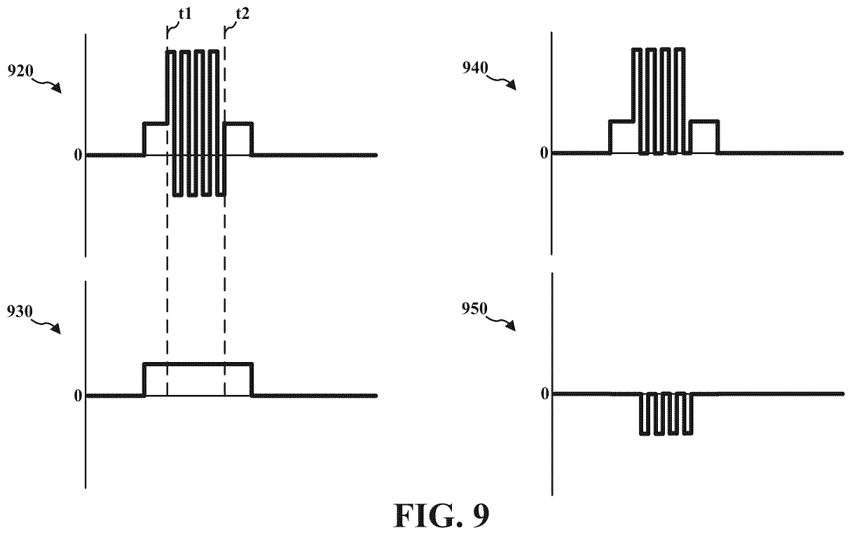

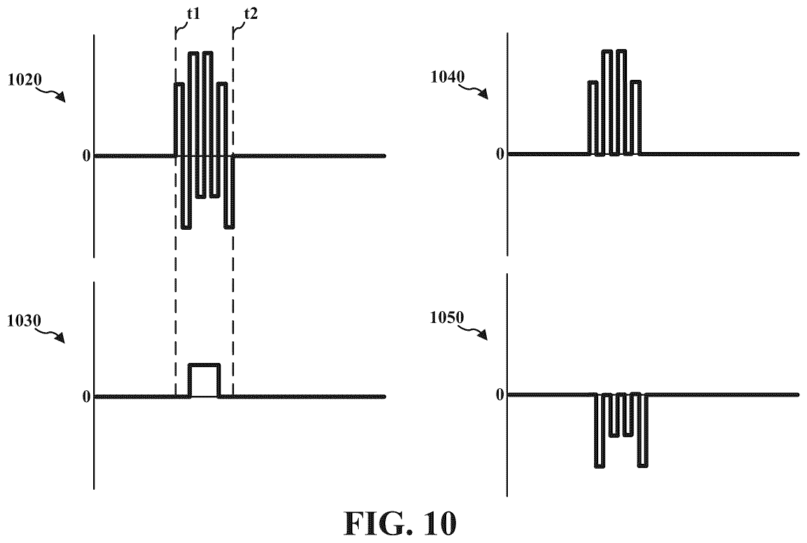

In some aspects, an offset component of a burst of transcutaneous current may have a longer duration or a shorter duration than the alternating current component. FIG. 9 is a diagram illustrating a burst of transcutaneous current 920 including an offset component 930 with a net positive sum of charge that begins before the first time t1 and ends after the first time t2. The corresponding first channel current 940 delivers a larger magnitude of charge than the corresponding second channel current 950 based on the offset component 930. FIG. 10 is a diagram illustrating a burst of transcutaneous current 1020 including an offset component 1030 with a net positive charge that begins after the first time t1 and ends before the first time t2. The corresponding first channel current 1040 delivers a larger magnitude of charge than the corresponding second channel current 1050 based on the offset component 1030.

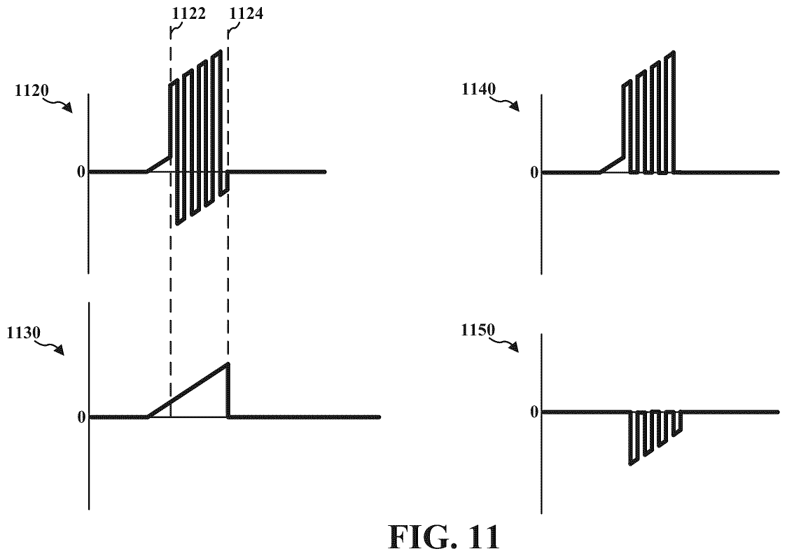

In some aspects, as illustrated in FIGS. 4 and 5, an offset component of a burst of transcutaneous current may be a burst of positive or negative DC current (e.g., a square pulse). In some aspects, the offset component may have a different shape. For example, FIG. 11 is a diagram illustrating a burst of transcutaneous current 1120 including an offset component 1130 with a net positive charge. The offset component 1130 is a ramp pulse beginning before the first time t1 and ending at the second time t2. The corresponding first channel current 1140 delivers a larger magnitude of charge than the corresponding second channel current 1150 based on the offset component 1130.

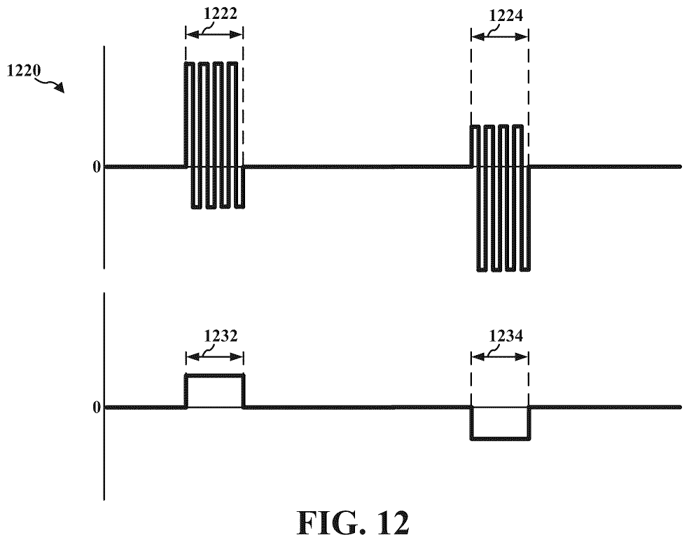

FIG. 12 is a diagram illustrating bursts of transcutaneous current including charge balanced offset components. Charge balanced transcutaneous current has a zero net sum of current. When current which is not charged balanced is applied to tissue, it may build up charge in the tissue and activate or cause sensation in the tissue. This may be desired at some locations (e.g., at the output electrodes of an implantable device) but may not be desired at other locations (e.g., at the receiving electrode of the implantable device or in the tissue path for transcutaneous current that does not pass through the implantable device).

An external generator may apply transcutaneous current 1220 to the skin of a subject. The transcutaneous current 1220 includes a first burst of transcutaneous current 1222 and a second burst of transcutaneous current 1224. The first burst of transcutaneous current 1222 and the second burst of transcutaneous current 1224 include corresponding alternating current components, such as the alternating current components of the set of alternating current components 420 described with respect to FIG. 4. The alternating current components are charge balanced. The first burst of transcutaneous current 1222 includes a first offset component 1232 and the second burst of transcutaneous current 1224 includes a second offset component 1234.

The first burst of transcutaneous current 1222 is not charged balanced on its own. It delivers a positive net sum of charge to tissue which the entire first burst of transcutaneous current 1222 passes through. Similarly, the second burst of transcutaneous current 1224 is not charge balanced on its own, and delivers a negative net sum of charge to tissue which the entire second burst of transcutaneous current 1234 passes through.

The first offset component 1232 and the second offset component 1234 are charge balanced with one another. The first offset component 1232 has a positive net sum of charge, and the second offset component 1234 has a negative net sum of charge of the same magnitude. Accordingly, although the first burst of transcutaneous current 1222 and the second burst of transcutaneous current 1224 are not charged balanced on their own, they are charge balanced when applied together. Tissue which the entire first burst of transcutaneous current 1222 and the entire second burst of transcutaneous current 1224 passes through may see a zero net sum of charge delivered to the tissue, which may reduce or prevent unwanted activation or sensation in that tissue.

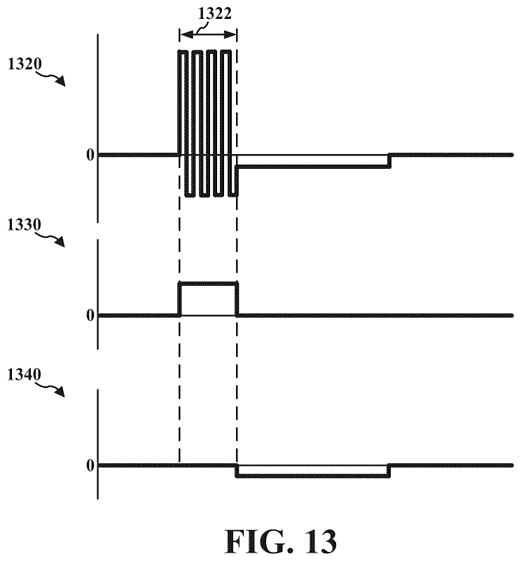

FIG. 13 is a diagram illustrating transcutaneous current with a balancing current. An external generator may apply transcutaneous current 1320 to the skin of a subject. The transcutaneous current 1320 includes a burst of transcutaneous current 1322 with an alternating current component and an offset component 1330. The transcutaneous current also includes a balancing current 1340.

The balancing current 1340) may be current having a net sum of charge having an opposite polarity to that of the offset component 1330. The offset component 1330 and the balancing current 1340 are charge balanced with one another. The offset component 1330 has a positive net sum of charge and the balancing current 1340 has a negative net sum of charge of the same magnitude. In some aspects, the balancing current 1340 may not be applied during the duration of the alternating current component or the duration of the offset component. In some aspects, the balancing current 1340 may have a longer duration than the offset component 1330 and may have a lower magnitude than the offset component 1330 such that the same magnitude of charge is delivered over a longer period of time. Applying the balancing component 1340 outside the duration of the burst of transcutaneous stimulation 1322 and/or applying the balancing current 1340 over a longer duration may result in the transcutaneous current 1320 being charged balanced without greatly impacting the disparity in charge delivered by the first channel current and charge delivered by the second channel current introduced by the offset component 1340.

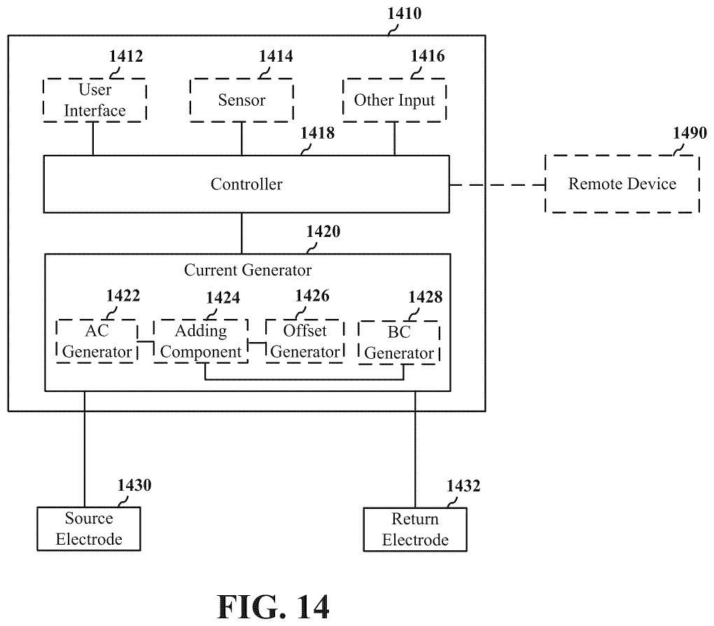

FIG. 14 is a block diagram illustrating an external generator 1410. For example, the external generator 1410) may be the external generator 110 of FIG. 1. The external generator 1410 includes a controller 1418, a current generator 1420, a source electrode 1430, and a return electrode 1432. The external generator 1410 may apply bursts of transcutaneous current to the skin of a subject, and one or more implantable device beneath the skin of the subject may present an electrical path which allows a portion of the applied bursts of transcutaneous current to path through the implantable device.

The current generator 1420 is coupled to the active electrode 1430 and the return electrode 1432. The current generator 1420 may include a constant current source or a constant voltage source. The current generator 1420 generates the current to be applied to the skin of the subject as transcutaneous current and applies the current between the active electrode 1430 and the return electrode 1432. The current generator 1420 may generate bursts of transcutaneous current including alternating current components and offset components, as discussed above.

In some aspects, the current generator 1420) may include an alternating current generator component 1422, an offset generator component 1426, and an adding component 1424. The alternating current generator component 1422 may generate the alternating current components of the bursts of transcutaneous current (e.g., as discussed above with respect to 420 of FIG. 4). The offset generator component 1422 may generate the offset components of the bursts of transcutaneous current (e.g., as discussed above with respect to 430 of FIG. 4, 630 of FIG. 6, 730 of FIG. 7, 830 of FIG. 8, 930 of FIG. 9, 1030 of FIG. 10, 1130 of FIG. 11, 1232 and 1234 of FIG. 12, and/or 1330 of FIG. 13). The alternating current generator component 1422 and the offset generator component 1426 may be coupled to the adding component 1424. The adding component 1424 may include an adder circuit. The adding component 1424 may receive the alternating current components from the alternating current component 1422, may receive the offset components from the offset generator component 1426, and may add them together to generate the bursts of transcutaneous current. The current generator 1420 may apply the bursts of transcutaneous current between the source electrode and the return electrode 1432.

In some aspects, the current generator 1420 includes a balancing current generator 1428. The balancing current generator 1428 may generate balancing current to charge balance the bursts of transcutaneous current (e.g., as described above with respect to 1340 of FIG. 13). The balancing current generator 1428 may be coupled to the adding component 1424. The adding component 1424 may add the balancing current to the bursts of transcutaneous current to generate the current applied between the source electrode 1430 and the return electrode 1432.

In some embodiments, the current generator 1420 may not include distinct alternating current generator, offset generator, balancing current generator, and adding components. The current generator 1420 may directly generate a burst of transcutaneous current with an alternating current component and an offset component (e.g., may not generate them as separate components and add them).

The controller 1418 may be coupled to the current generator 1420. In some aspects, the current generator 1420 may be preconfigured to apply bursts of transcutaneous current with preconfigured offset components. In some aspects, the controller 1418 may control the current generator 1420 to set the shape, amplitude, timing, and/or other characteristics of the offset components of the transcutaneous bursts generated by the current generator 1420.

In some aspects, the external generator 1410 includes a user interface 1412 coupled to the controller 1418. The controller 1418 may receive user inputs from the user interface 1412. The controller 1418 may control the current generator 1420 to set the shape, amplitude, timing, and/or other characteristics of the offset components based on the user inputs. In some aspects, the controller 1418 may receive a user input indicating that more current should be delivered to the anodic channel of the implantable device and may control the current generator 1420 to make the offset more positive (e.g., increase a positive amplitude or decrease a negative amplitude of the offset). The controller 1418 may receive a user input indicating that more current should be delivered to the cathodic channel of the implantable device and may control the current generator 1420 to make the offset more negative (e.g., decrease a positive amplitude or increase a negative amplitude of the offset). The controller 1418 may also control the current generator 1420 to set the shape, amplitude, frequency, and/or other characteristics of the alternating current components based on the user inputs. For example, the controller 1418 may receive a user input indicating that more current should be delivered to both the anodic channel and the cathodic channel, or that less current should be delivered to both the anodic and the cathodic channel and the controller 1418 may control the current generator 1420 to increase or decrease the amplitude of the alternating current components.

In some aspects, the controller 1418 may receive a first user input identifying a first level of current for a first channel and may receive a second user input identifying a second level of current for a second channel. The controller 1418 may control the current generator 1420 to set the net charge of the offset components (e.g., through shape or amplitude) and to set the amplitude of the alternating current components based on the first user input and the second user input. For example, the level of the current identified by the first user input may be referred to as L1 and the level of current identified by the second user input may be referred to as L2. The controller 1418 may utilize the formula (L1+L2)/2 to determine a value for the amplitude of the alternating current component, may utilize the formula (L1−L2)/2 to determine a value for the net charge of the offset component, and may control the current generator 1420 to set the amplitude of the alternating current components and the net charge of the offset components based on those values.

In some aspects, the external generator 1410 includes one or more sensor 1414 coupled to the controller 1418. The controller 1418 may receive sensor inputs from the sensor 1414. In some aspects, the external generator 1410 may receive sensor inputs from an external sensor transmitted to the external generator 1410 (e.g., from an implanted sensor, either as part of the implantable device or independent from the implantable device). The controller 1418 may control the current generator 1420 to set the shape, amplitude, timing, and/or other characteristics of the offset components based on the user inputs, and/or may control the current generator 1420 to set the shape, amplitude frequency, and/or other characteristics of the alternating current components based on the sensor inputs. The sensor 1414 may be an EMG sensor, an evoked potentials sensor, an accelerometer, a gyroscope, a temperature sensor, a goniometer, a pressure sensor, a chemical sensor, an oxygen sensor, or a skin impedance sensor.

In some aspects, the external generator 1410 includes one or more other input 1416 coupled to the controller 1418, and the controller 1418 may control the current generator 1420 to set the characteristics of the offset components and/or the characteristics of the alternating current components based on input from the other inputs 1416. For example, the other input 1416 may be a clock circuit, and the controller 1418 may set the characteristics of the offset components and/or the alternating current components based on the time of day (e.g., may increase or decrease current delivered to both channels or to a particular channel at night).

In some aspects, the external generator 1410 may be coupled (e.g., may wirelessly communicate with) a remote device 1490. In some aspects, the remote device 1090 may include communication components capable of communicating to a cloud-based system, for example, for storage or tracking of patient treatment data. The remote device 1490 may include a user interface and may transmit user input to the controller 1418, may include one or more sensor and may transmit sensor input to the controller 1418, and may transmit other input to the controller 1418. The controller 1418 may control current generator 1420 to set the characteristics of the offset components and/or the alternating current components based on the user input, sensor input, or other input received from the remote device 1490.

By way of example, one or more component of the block diagram discussed above (e.g., the controller 1418), or any portion of a component, or any combination of components may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure. One or more processors in the processing system may be coupled to a computer readable medium storing software and may execute the software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

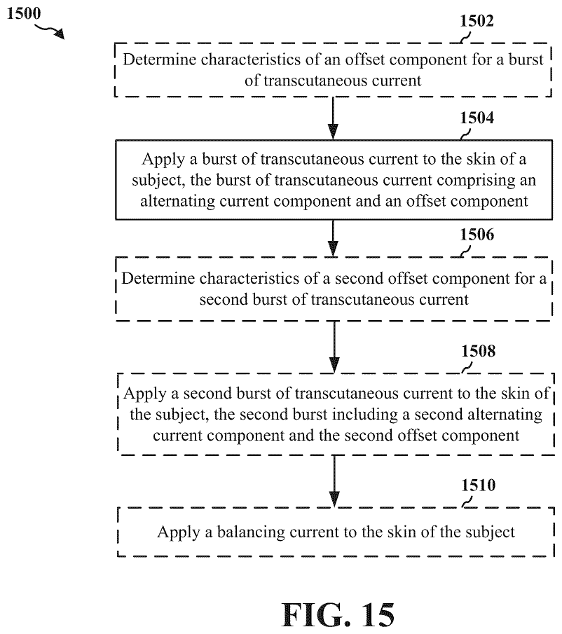

FIG. 15 is a flowchart illustrating a method of delivering transcutaneous current to tissue of a subject through a first channel and a second channel of one or more implantable device. The method may be performed by an external generator (e.g., the external generator 110 of FIG. 1 or the external generator 1410 of FIG. 14). The one or more implantable device may be implanted beneath the skin of the subject. In some aspects, one implantable device includes both the first stimulation channel and the second stimulation channel. In some other aspects, the first channel and the second channel are channels in separate implantable devices—the first channel is included in a first implantable device and the second channel is included in a second implantable device.

In some aspects, at 1502, the external generator may determine characteristics of an offset component for a burst of transcutaneous current. In some aspects, the external generator may receive an input from a user interface or a sensor input from a sensor, wherein a characteristic of the offset component is based on the input. The characteristic may be an amplitude, a shape, a timing, or a duration of the offset component.

In some aspects, the external generator may receive a first input corresponding to a first current level for the first channel and may receive a second input corresponding to a second current level for the second channel. A characteristic of the offset component and a characteristic of the alternating current component may be based on the first input and the second input. The characteristic of the alternating current component may be an amplitude of the alternating current component and the characteristic of the offset component may be an amplitude, a shape, or a duration of the offset component.

At 1504, the external generator may apply a burst of transcutaneous current to the skin of the subject, the burst of transcutaneous current including an alternating current component and an offset component. In some aspects, the offset component may have the characteristics determined at 1502. A portion of the burst of transcutaneous current passes through the one or more implantable device implanted beneath the skin of the subject. The first channel comprises a rectifier allowing positive current to pass through the first channel. The second channel comprises a rectifier allowing negative current to pass through the second channel. The burst of transcutaneous current delivers a first electrical charge to tissue at an output electrode of the first channel based on the offset component, and the burst of transcutaneous current delivers a second electrical charge different than the first electrical charge to tissue at an output electrode of the second channel based on the offset component. In some aspects, the first electrical charge activates the tissue (e.g., the first electrical charge causes a difference between an intracellular and an extracellular electrical potential of the tissue, resulting in activation of the tissue) at the output electrode of the first channel and the second electrical charge does not activate the tissue at the output electrode of the second channel. In some aspects, the first electrical charge activates the tissue at the output electrode of the first channel, the second electrical charge activates the tissue at the output electrode of the second channel, and the activation level of the tissue at the output electrode of the first channel is different than the activation level of the tissue at the output electrode of the second channel.

In some aspects, the offset component is applied for the entire duration of the alternating current component. In some aspects, the offset component is applied for a subset of the duration of the alternating current component. In some aspects, the offset component is applied before the alternating current component. In some aspects, the offset component is applied after the alternating current component.

At 1506, the external generator may determine characteristics of a second offset component for a second burst of transcutaneous current. For example, the characteristics of the second offset component may be determined as described above at 1502 with respect to the first offset component.

At 1508, the external generator may apply a second burst of transcutaneous current to the skin of the subject, the second burst of transcutaneous current including a second alternating current component and a second offset component. In some aspects, the offset component may have the characteristics determined at 1502. The second burst of transcutaneous current delivers a third electrical charge to the tissue at the output electrode of the first channel based on the second offset component. The second burst of transcutaneous current delivers a fourth electrical charge to the tissue at the output electrode of the second channel based on the second offset component. The first electrical charge is greater than the second electrical charge, and the fourth electrical charge is greater than the third electrical charge. In some aspects, the offset component and the second offset component are charge balanced.

At 1510, the external generator may apply a balancing current to the skin of the subject. The balancing current may not be applied during the burst of transcutaneous current (e.g., may be applied outside the time during which the burst of transcutaneous current is applied. In some aspects, the balancing current has a longer duration than the offset component.

It is understood that the specific order or hierarchy of blocks in the processes/flowcharts disclosed is an illustration of example approaches. Based upon design preferences, it is understood that the specific order or hierarchy of blocks in the processes/flowcharts may be rearranged. Further, some blocks may be combined or omitted. The accompanying method claims present elements of the various blocks in a sample order, and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

### LISTING OF EMBODIMENTS

Various exemplary embodiments of the present disclosure are further illustrated by the following listing of embodiments, which should not be construed to unduly limit the present disclosure:

1. A method of delivering transcutaneous current to tissue of a subject through a first channel and a second channel of one or more implantable device implanted beneath the skin of the subject, the method comprising: applying a burst of transcutaneous current to the skin of the subject, the burst of transcutaneous current comprising an alternating current component and an offset component, wherein a portion of the burst of transcutaneous current passes through the one or more implantable device implanted beneath the skin of the subject, the first channel comprises a rectifier allowing positive current to pass through the first channel, the second channel comprises a rectifier allowing negative current to pass through the second channel, the burst of transcutaneous current delivers a first electrical charge to tissue at an output electrode of the first channel based on the offset component, and the burst of transcutaneous current delivers a second electrical charge different than the first electrical charge to tissue at an output electrode of the second channel based on the offset component.

2. A method according to embodiment 1, wherein the first electrical charge activates the tissue at the output electrode of the first channel and the second electrical charge does not activate the tissue at the output electrode of the second channel.

3. A method according to any of embodiments 1 to 2, wherein the first electrical charge activates the tissue at the output electrode of the first channel, the second electrical charge activates the tissue at the output electrode of the second channel, and wherein the activation level of the tissue at the output electrode of the first channel is different than the activation level of the tissue at the output electrode of the second channel.

4. A method according to any of embodiments 1 to 3, wherein the offset component is applied for the entire duration of the alternating current component.

5. A method according to any of embodiments 1 to 4, wherein the offset component is applied for a subset of the duration of the alternating current component.

6. A method according to any of embodiments 1 to 5, wherein the offset component is applied before the alternating current component.

7. A method according to any of embodiments 1 to 6, wherein the offset component is applied after the alternating current component.

8. A method according to any of embodiments 1 to 7, wherein one implantable device comprises the first stimulation channel and the second stimulation channel.

9. A method according to any of embodiments 1 to 8, wherein a first implantable device comprises the first channel and a second implantable device comprises the second channel.

10. A method according to any of embodiments 1 to 9, further comprising: receiving a first input corresponding to a first current level for the first channel; and receiving a second input corresponding to a second current level for the second channel, wherein a characteristic of the offset component and a characteristic of the alternating current component are based on the first input and the second input.

11. A method according to any of embodiments 1 to 10, wherein the characteristic of the alternating current component is an amplitude of the alternating current component and the characteristic of the offset component is an amplitude, a shape, or a duration of the offset component.

12. A method according to any of embodiments 1 to 11, further comprising: applying a second burst of transcutaneous current to the skin of the subject, the second burst of transcutaneous current comprising a second alternating current component and a second offset component, wherein the second burst of transcutaneous current delivers a third electrical charge to the tissue at the output electrode of the first channel based on the second offset component, the second burst of transcutaneous current delivers a fourth electrical charge to the tissue at the output electrode of the second channel based on the second offset component, the first electrical charge is greater than the second electrical charge, and the fourth electrical charge is greater than the third electrical charge.

13. A method according to any of embodiments 1 to 12, wherein the offset component and the second offset component are charge balanced.

14. A method according to any of embodiments 1 to 13, further comprising: applying a balancing current to the skin of the subject, wherein the balancing current is charge balanced with the offset component.

15. A method according to any of embodiments 1 to 14, wherein the balancing current is not applied during the burst of transcutaneous current.

16. A method according to any of embodiments 1 to 15, wherein the balancing current has a longer duration than the offset component.

17. A method according to any of embodiments 1 to 16, further comprising: receiving an input from a user interface or a sensor, wherein a characteristic of the offset component is based on the input.

18. A method according to any of embodiments 1 to 17, wherein the characteristic is an amplitude, a shape, a timing, or a duration of the offset component.

19. A method according to any of embodiments 1 to 18, wherein the burst of transcutaneous stimulation is asymmetrical with respect to the amplitude of the burst based on the offset component.

20. An external generator, comprising: a current generator; a first skin electrode coupled to the current generator; and a second skin electrode coupled to the current generator, wherein the current generator is configured to apply a burst of transcutaneous current between the first skin electrode and the second skin electrode to a skin of a subject, the burst of transcutaneous current comprising an alternating current component and an offset component, a portion of the burst of transcutaneous current passes through one or more implantable device implanted beneath the skin of the subject, the one or more implantable device including a first channel and a second channel, the first channel of the implantable device comprises a rectifier allowing positive current to pass through the first channel, the second channel comprises a rectifier allowing negative current to pass through the second channel, the burst of transcutaneous current delivers a first electrical charge to tissue at an output electrode of the first channel based on the offset component, and the burst of transcutaneous current delivers a second electrical charge different than the first electrical charge to tissue at an output electrode of the second channel based on the offset component.

21. An external generator according to embodiment 20, wherein the first electrical charge activates the tissue at the output electrode of the first channel and the second electrical charge does not activate the tissue at the output electrode of the second channel.

22. An external generator according to any of embodiments 20 to 21, wherein the first electrical charge activates the tissue at the output electrode of the first channel, the second electrical charge activates the tissue at the output electrode of the second channel, and wherein the activation level of the tissue at the output electrode of the first channel is different than the activation level of the tissue at the output electrode of the second channel.

23. An external generator according to any of embodiments 20 to 22, wherein the current generator is configured to apply the offset component for the entire duration of the alternating current component.

24. An external generator according to any of embodiments 20 to 23, wherein the current generator is configured to apply the offset component for a subset of the duration of the alternating current component.

25. An external generator according to any of embodiments 20 to 24, wherein the current generator is configured to apply the offset component before the alternating current component.

26. An external generator according to any of embodiments 20 to 25, wherein the current generator is configured to apply the offset component after the alternating current component.

27. An external generator according to any of embodiments 20 to 26, wherein one implantable device comprises the first stimulation channel and the second stimulation channel.

28. An external generator according to any of embodiments 20 to 27, wherein a first implantable device comprises the first channel and a second implantable device comprises the second channel.

29. An external generator according to any of embodiments 20 to 28, further comprising a controller configured to: receive a first input corresponding to a first current level for the first channel; and receive a second input corresponding to a second current level for the second channel, wherein a characteristic of the offset component applied by the current generator and a characteristic of the alternating current component applied by the current generator based on the first input and the second input.

30. An external generator according to any of embodiments 20 to 29, wherein the characteristic of the alternating current component is an amplitude of the alternating current component and the characteristic of the offset component is an amplitude, a shape, or a duration of the offset component.

31. An external generator according to any of embodiments 20 to 30, wherein the current generator is further configured to apply a second burst of transcutaneous current to the skin of the subject, the second burst of transcutaneous current comprising a second alternating current component and a second offset component, wherein the second burst of transcutaneous current delivers a third electrical charge to the tissue at the output electrode of the first channel based on the second offset component, the second burst of transcutaneous current delivers a fourth electrical charge to the tissue at the output electrode of the second channel based on the second offset component, the first electrical charge is greater than the second electrical charge, and the fourth electrical charge is greater than the third electrical charge.

32. An external generator according to any of embodiments 20 to 31, wherein the offset component and the second offset component are charge balanced.

33. An external generator according to any of embodiments 20 to 32, wherein the current generator is further configured to apply a balancing current to the skin of the subject, wherein the balancing current is charge balanced with the offset component.

34. An external generator according to any of embodiments 20 to 33, wherein the current generator does not apply the balancing current during the burst of transcutaneous current.

35. An external generator according to any of embodiments 20 to 34, wherein the balancing current has a longer duration than the offset component.

36. An external generator according to any of embodiments 20 to 35, further comprising a controller configured to receive an input from a user interface or a sensor, wherein a characteristic of the offset component is based on the input.

37. An external generator according to any of embodiments 20 to 36, wherein the characteristic is an amplitude, a shape, a timing, or a duration of the offset component.

38. An external generator according to any of embodiments 20 to 37, wherein the burst of transcutaneous stimulation is asymmetrical with respect to the amplitude of the burst based on the offset component.

What is claimed is:

1. A method of delivering transcutaneous current to tissue of a subject through a first channel and a second channel of one or more implantable device implanted beneath the skin of the subject, the method comprising:
applying a burst of transcutaneous current to the skin of the subject, the burst of transcutaneous current comprising an alternating current component and an offset component, wherein
a portion of the burst of transcutaneous current passes through the one or more implantable device implanted beneath the skin of the subject,
the first channel comprises a rectifier allowing positive current to pass through the first channel,
the second channel comprises a rectifier allowing negative current to pass through the second channel,
the burst of transcutaneous current delivers a first electrical charge to tissue at an output electrode of the first channel based on the offset component, and
the burst of transcutaneous current delivers a second electrical charge different than the first electrical charge to tissue at an output electrode of the second channel based on the offset component.

2. The method of claim 1, wherein the first electrical charge activates the tissue at the output electrode of the first channel and the second electrical charge does not activate the tissue at the output electrode of the second channel.

3. The method of claim 1, wherein the first electrical charge activates the tissue at the output electrode of the first channel, the second electrical charge activates the tissue at the output electrode of the second channel, and wherein the activation level of the tissue at the output electrode of the first channel is different than the activation level of the tissue at the output electrode of the second channel.

4. The method of claim 1, wherein the offset component is applied for the entire duration of the alternating current component.

5. The method of claim 1, wherein the offset component is applied for a subset of the duration of the alternating current component.

6. The method of claim 1, wherein the offset component is applied before the alternating current component.

7. The method of claim 1, wherein the offset component is applied after the alternating current component.

8. The method of claim 1, wherein one implantable device comprises the first stimulation channel and the second stimulation channel.

9. The method of claim 1, wherein a first implantable device comprises the first channel and a second implantable device comprises the second channel.

10. The method of claim 1, further comprising:
receiving a first input corresponding to a first current level for the first channel; and
receiving a second input corresponding to a second current level for the second channel,
wherein a characteristic of the offset component and a characteristic of the alternating current component are based on the first input and the second input.

11. The method of claim 10, wherein the characteristic of the alternating current component is an amplitude of the alternating current component and the characteristic of the offset component is an amplitude, a shape, or a duration of the offset component.

12. The method of claim 1, further comprising:
applying a second burst of transcutaneous current to the skin of the subject, the second burst of transcutaneous current comprising a second alternating current component and a second offset component, wherein
the second burst of transcutaneous current delivers a third electrical charge to the tissue at the output electrode of the first channel based on the second offset component,
the second burst of transcutaneous current delivers a fourth electrical charge to the tissue at the output electrode of the second channel based on the second offset component,
the first electrical charge is greater than the second electrical charge, and
the fourth electrical charge is greater than the third electrical charge.

13. The method of claim 12, wherein the offset component and the second offset component are charge balanced.

14. The method of claim 1, further comprising:
applying a balancing current to the skin of the subject, wherein the balancing current is charge balanced with the offset component.

15. The method of claim 14, wherein the balancing current is not applied during the burst of transcutaneous current.

16. The method of claim 14, wherein the balancing current has a longer duration than the offset component.

17. The method of claim 1, further comprising:
receiving an input from a user interface or a sensor, wherein a characteristic of the offset component is based on the input.

18. The method of claim 17, wherein the characteristic is an amplitude, a shape, a timing, or a duration of the offset component.

19. The method of claim 1, wherein the burst of transcutaneous stimulation is asymmetrical with respect to the amplitude of the burst based on the offset component.

20. An external generator, comprising:
a current generator;
a first skin electrode coupled to the current generator; and
a second skin electrode coupled to the current generator,
wherein the current generator is configured to apply a burst of transcutaneous current between the first skin electrode and the second skin electrode to a skin of a subject, the burst of transcutaneous current comprising an alternating current component and an offset component,
a portion of the burst of transcutaneous current passes through one or more implantable device implanted beneath the skin of the subject, the one or more implantable device including a first channel and a second channel,
the first channel of the implantable device comprises a rectifier allowing positive current to pass through the first channel,
the second channel comprises a rectifier allowing negative current to pass through the second channel,
the burst of transcutaneous current delivers a first electrical charge to tissue at an output electrode of the first channel based on the offset component, and
the burst of transcutaneous current delivers a second electrical charge different than the first electrical charge to tissue at an output electrode of the second channel based on the offset component.

21. The external generator of claim 20, wherein the first electrical charge activates the tissue at the output electrode of the first channel and the second electrical charge does not activate the tissue at the output electrode of the second channel.

22. The external generator of claim 20, wherein the first electrical charge activates the tissue at the output electrode of the first channel, the second electrical charge activates the tissue at the output electrode of the second channel, and wherein the activation level of the tissue at the output electrode of the first channel is different than the activation level of the tissue at the output electrode of the second channel.

23. The external generator of claim 20, wherein the current generator is configured to apply the offset component for the entire duration of the alternating current component.

24. The external generator of claim 20, wherein the current generator is configured to apply the offset component for a subset of the duration of the alternating current component.

25. The external generator of claim 20, wherein the current generator is configured to apply the offset component before the alternating current component.

26. The external generator of claim 20, wherein the current generator is configured to apply the offset component after the alternating current component.

27. The external generator of claim 20, wherein one implantable device comprises the first stimulation channel and the second stimulation channel.

28. The external generator of claim 20, wherein a first implantable device comprises the first channel and a second implantable device comprises the second channel.

29. The external generator of claim 20, further comprising a controller configured to:
receive a first input corresponding to a first current level for the first channel; and
receive a second input corresponding to a second current level for the second channel,
wherein a characteristic of the offset component applied by the current generator and a characteristic of the alternating current component applied by the current generator based on the first input and the second input.

30. The external generator of claim 29, wherein the characteristic of the alternating current component is an amplitude of the alternating current component and the characteristic of the offset component is an amplitude, a shape, or a duration of the offset component.

31. The external generator of claim 20, wherein the current generator is further configured to apply a second burst of transcutaneous current to the skin of the subject, the second burst of transcutaneous current comprising a second alternating current component and a second offset component, wherein
the second burst of transcutaneous current delivers a third electrical charge to the tissue at the output electrode of the first channel based on the second offset component,
the second burst of transcutaneous current delivers a fourth electrical charge to the tissue at the output electrode of the second channel based on the second offset component,
the first electrical charge is greater than the second electrical charge, and
the fourth electrical charge is greater than the third electrical charge.

32. The external generator of claim 31, wherein the offset component and the second offset component are charge balanced.

33. The external generator of claim 20, wherein the current generator is further configured to apply a balancing current to the skin of the subject, wherein the balancing current is charge balanced with the offset component.

34. The external generator of claim 33, wherein the current generator does not apply the balancing current during the burst of transcutaneous current.

35. The external generator of claim 33, wherein the balancing current has a longer duration than the offset component.

36. The external generator of claim 20, further comprising a controller configured to receive an input from a user interface or a sensor, wherein a characteristic of the offset component is based on the input.

37. The external generator of claim 36, wherein the characteristic is an amplitude, a shape, a timing, or a duration of the offset component.

38. The external generator of claim 20, wherein the burst of transcutaneous stimulation is asymmetrical with respect to the amplitude of the burst based on the offset component.
